(19) United States
(12) Patent Application Publication
(10) Pub. No.: US 2007/0099170 A1
(43) Pub. Date: May 3, 2007

(54) Method for treatment and storage of blood and blood products using endogenous alloxazines and acetate

(75) Inventors: Raymond P. Goodrich, Lakewood, CO (US); Junzhi Li, Gold River, CA (US)

Correspondence Address: GAMBRO, INC;PATENT DEPARTMENT, 10810 W COLLINS AVE, LAKEWOOD, CO, 80215, US

(73) Assignee: Navigant Biotechnologies, Inc., Lakewood, CO (US)

(21) Appl. No.: 11/607,737

(22) Filed: Dec. 1, 2006

Related U.S. Application Data
(63) Continuation-in-part of application No. 10/377,524, filed on Feb. 28, 2003.
Application No. 10/377,524 is a continuation of application No. 09/586,147, filed on Jun. 2, 2000, now abandoned.
Application No. 09/586,147 is a continuation-in-part of application No. 09/357,188, filed on Jul. 20, 1999, now Pat. No. 6,277,337.
Application No. 09/357,188 is a continuation-in-part of application No. 09/119,666, filed on Jul. 21, 1998, now Pat. No. 6,258,577.
(60) Provisional application No. 60/597,506, filed on Dec. 6, 2005.

(51) Int. Cl. A01N 1/02; C12N 5/08

(52) U.S. Cl. 435/2; 435/372

(57) ABSTRACT

Methods are provided for treatment and storage of blood and blood products using at least endogenous alloxazines and acetate. Methods include adding a blood component additive solution comprising at least an endogenous alloxazine and acetate to a fluid comprising at least one collected blood component.

### CROSS-REFERENCE TO RELATED APPLICATIONS

This application is a continuation-in-part of U.S. application Ser. No. 10/377,524 filed Feb. 28, 2003, which is a continuation of U.S. application Ser. No. 09/586,147 filed Jun. 2, 2000, now abandoned, which is a continuation-in-part of U.S. application Ser. No. 09/357,188, now U.S. Pat. No. 6,277,337, filed Jul. 20, 1999 which is a continuation-in-part of U.S. application Ser. No. 09/119,666, now U.S. Pat. No. 6,258,577, filed Jul. 21, 1998. This application also claims the priority of U.S. provisional application No. 60/597,506 filed on Dec. 6, 2005.

### FIELD OF THE INVENTION

The invention generally relates to synthetic media for use in the collection and/or storage of platelets intended for in vivo use, including synthetic media used in conjunction with the pathogen reduction of platelets.

### BACKGROUND

Whole blood collected from volunteer donors for transfusion recipients is typically separated into its components: red blood cells, white blood cells, platelets, and plasma using various known methods. Each of these fractions are individually stored under conditions specific to each blood component, and used to treat a multiplicity of specific conditions and disease states. For example, the red blood cell component is used to treat anemia, the concentrated platelet component is used to control bleeding, and the plasma component is used frequently as a source of blood proteins such as clotting factors.

In the blood banking area, contamination of blood supplies with infectious microorganisms such as HIV, hepatitis and other viruses and bacteria presents a serious health hazard for those who must receive transfusions of whole blood or administration of various blood components. Blood screening procedures may miss contaminants, and sterilization procedures which do not damage cellular blood components but effectively inactivate all infectious viruses and other microorganisms have not been previously available.

Another major issue in blood banking is the loss of function of the blood components during storage. Platelets in particular, need to be resuspended after separation from other blood components in either a suitable storage solution or in plasma to improve or at least maintain platelet quality during storage.

If platelets are stored in plasma, they are typically stored in concentrations of around 900-2100×10³/μL. A side effect of transfusing platelets with plasma is that the transfusion recipient may develop allergic reactions to components in the donor plasma and/or TRALI (Transfusion Related Acute Lung Injury.) Another consideration is one of cost. Plasma by itself can be used or sold in order to fractionate the plasma proteins into clotting factors and the like.

Therefore, it is desirable to store platelets in synthetic storage solutions. If platelets are stored in synthetic storage solutions, they are also typically stored in concentrations of around 900-2100×10³/μL. Several commercially available solutions include PASII (available from MacoPharma), PASII (available from Baxter) and CompoSol (available from Fresenius). The commercially available platelet storage solutions contain additives such as phosphate, glucose, sodium, potassium, citrate, magnesium, sulfate and acetate which are thought to enhance platelet metabolism during storage.

In order to maintain viability, platelets must continuously generate enough adenosine triphosphate (ATP) to meet their energy needs. Two pathways are normally available to generate ATP, the glycolysis pathway and the oxidative phosphorylation pathway. In glycolysis, one molecule of glucose is converted to two molecules of lactic acid to generate two molecules of ATP. In oxidative phosphorylation, glucose, fatty acids or amino acids enter the citric acid cycle and are converted to CO₂ and water. This pathway requires the presence of an adequate supply of oxygen to accept the protons produced by the breakdown of glucose. It is much more efficient than glycolysis. Oxidative metabolism of substrates to CO₂ and water yields 36 molecules of ATP.

It has been recognized that platelets will meet their energy needs in a manner which is not necessarily consistent with their long term storage in a viable condition. When given adequate oxygen, platelets produce most of their ATP through oxidation, but continue to produce lactic acid instead of diverting all metabolized glucose through the oxidative pathway. During the storage of platelets in plasma, lactic acid concentrations rise at approximately 2.5 mM per day. See Murphy et al.; “Platelet Storage at 22° C., Blood, 46(2): 209-218 (1975); Murphy, “Platelet Storage for Transfusion”, Seminars in Hematology, 22(3): 165-177 (1985). This leads to gradual fall in pH. As explained in the Murphy articles, when lactic acid reaches about 20 mM, the pH which started at 7.2 may reach 6.0. Since platelet viability is irreversibly lost if pH falls to 6.1 or below, a major limiting variable for platelet storage is pH.

Therefore, regulation of pH is a major factor in long-term platelet storage. Virtually all units of platelets show a decrease in pH from their initial value of approximately 7.0. This decrease is primarily due to the production of lactic acid by platelet glycolysis and to a lesser extent to accumulation of CO₂ from oxidative phosphorylation. As the pH falls, the platelets change shape from discs to spheres. If the pH falls to around 6.0, irreversible changes in platelet morphology and physiology render them non-viable after transfusion. An important goal in platelet preservation, therefore, is to prevent this decrease in pH.

In association with the decrease in pH, decreases in the total amount of ATP produced per platelet have been observed. The depletion of metabolically available ATP affects platelet function because ATP is essential for such roles as platelet adhesion and platelet aggregation. The ability of platelets to maintain total ATP at close to normal levels has been found to be associated with platelet viability during storage.

In designing a platelet storage medium, one solution to the above problems has been to include an additive which acts as both a substrate for oxidative phosphorylation and as a buffer to counteract the acidifying effect of the lactic acid which platelets produce during storage. Acetate has been found to be a suitable substrate. In addition, its oxidation produces bicarbonate:
CH₃ COOO+2O₂═CO₂+HCO₃+H₂O

Thus, the use of acetate serves two purposes, as a substrate for oxidative phosphorylation and as a buffer. Such platelet storage solutions disclosed in U.S. Pat. Nos. 5,344,752 and 5,376,524.

Another additive, which is a useful substrate in the storage of blood and blood components includes a compound which stimulates mitochondrial activity. One such suitable compound is endogenous 7,8-dimethyl-10-ribityl isoalloxazine (riboflavin), its metabolites and precursors. This mitochondrial stimulating compound may include endogenously-based derivatives which are synthetically derived analogs and homologs of riboflavin which may have or lack lower (1-5) alkyl or halogen substituents, and which preserve the function and substantial non-toxicity thereof. This is disclosed in U.S. patent application Ser. No. 10/430,896.

It is believed that these agents work to maintain platelet viability during storage by stimulating mitochondrial activity. FMN and FAD produced by metabolism of riboflavin are essential elements for electron transport activity. This activity is heavily involved in mitochondrial respiration. By providing elevated levels of riboflavin to cells, it is possible to enhance mitochondrial respiration and thus promote ATP production via oxidative phosphorylation rather than through glycolysis.

However, to date, no storage or additive solution exists which maintains platelet viability during storage or during a pathogen reduction treatment using a substrate which acts as a substrate for oxidative phosphorylation and as a buffer, in combination with a substrate which stimulates mitochondrial activity. It is to such a solution that the present invention is directed.

### SUMMARY

This invention is directed toward a blood component storage or additive solution containing at least a photosensitizer-like additive and acetate which may be used to collect, treat and/or store platelets.

This invention also is directed toward a method of pathogen reducing blood or a collected blood component which includes the steps of adding to the blood or blood component to be pathogen reduced an effective non-toxic amount of a mixture of an endogenous photosensitizer or endogenously-based derivative photosensitizer and acetate; and exposing the mixed fluid to photoradiation sufficient to activate the photosensitizer whereby at least some of the pathogens are inactivated.

### BRIEF DESCRIPTION OF THE FIGURES

FIG. 1 is a graph comparing the rate of glucose consumption of treated and untreated platelets stored for five and seven days.

FIG. 2 is a graph comparing the rate of lactate production of treated and untreated platelets stored for five and seven days.

FIG. 3 is a graph comparing pH change of treated and untreated platelets stored for five and seven days.

FIG. 4 is a graph comparing the rate of O₂ consumption by treated and untreated platelets stored over a seven day period.

FIG. 5 is a graph comparing the rate of CO₂ production by treated and untreated platelets stored over a seven day period.

FIG. 6 is a graph comparing the rate of bicarbonate neutralization by treated and untreated platelets stored over a seven day period.

FIG. 7 is a graph comparing the extent of platelet shape change in treated and untreated platelets stored over a seven day period.

FIG. 8 is a graph comparing the rate of glucose consumption by platelets stored over 12 days in a

FIG. 9 is a graph comparing the rate of lactate production by platelets stored over 12 days in a solution containing riboflavin and acetate with platelets stored in saline.

FIG. 10 is a graph comparing the cell counts of platelets stored over 12 days in a solution containing riboflavin and acetate with platelets stored in saline.

FIG. 11 shows an embodiment of this invention using a series of bags to flow the photosensitizer and additive into the blood components to be pathogen reduced.

FIG. 12 shows an embodiment of this invention using a blood bag to contain the fluid being pathogen reduced while exposing the fluid to photoradiation from a light source.

### DETAILED DESCRIPTION

The invention generally relates to a storage and treatment solution for use with blood components intended for in vivo use.

As discussed above, a platelet storage solution which contains acetate and riboflavin may greatly increase platelet viability during long term storage. The pH of such solution is preferably between about 5.0 and 7.4. Such a solution may be useful as a carrier for platelet concentrates to allow maintenance of cell quality and metabolism during storage, allow for a reduction in the amount of plasma in the stored platelets and extend storage life. These solutions also allow the residual plasma in platelet concentrates to be reduced to around 20-60 mLs/10¹¹ cells compared with a standard level of around 75-100 mLs/10¹¹ cells.

There are other factors besides long term storage which might cause platelets to enter glycolysis and thereby accumulate lactic acid. One example of an external treatment which might cause platelets to accumulate lactate is a procedure to inactivate or reduce any pathogens which might be contained in or around the cells to be transfused into a recipient. Currently used methods to reduce pathogenic contaminants which may be present in blood components may cause damage to the mitochondria of the cells being treated. Ultraviolet light for instance, has been shown to damage mitochondria. If mitochondria are damaged, cells can only make ATP through the glycolysis pathway, causing a buildup of lactic acid in the cell, and a subsequent drop in pH during storage.

The present invention therefore also contemplates a solution which can be used in a procedure to reduce any pathogens which may be contained in the whole blood or collected blood components. In this embodiment, an additive that behaves as a photosensitizer if exposed to light is selectively employed to help eliminate contaminating pathogens. The pathogen reduction solution may also contain an additive such as acetate that acts as a substrate for oxidative phosphorylation, to help maintain cell viability of the cells during and/or after the pathogen reduction procedure.

If pathogen reduction of blood and/or blood components is desired, additives which act as photosensitizers upon exposure to light are useful in this invention. Such additives include endogenous photosensitizers. Examples of such endogenous photosensitizers are alloxazines such as 7,8-dimethyl-10-ribityl isoalloxazine (riboflavin), 7,8,10-trimethylisoalloxazine (lumiflavin), 7,8-dimethylalloxazine (lumichrome), isoalloxazine-adenine dinucleotide (flavin adenine dinucleotide [FAD]), alloxazine mononucleotide (also known as flavin mononucleotide [FMN] and riboflavin-5-phosphate), their metabolites and precursors. When endogenous photosensitizers are used, particularly when such photosensitizers are not inherently toxic or do not yield toxic photoproducts after photoradiation, no removal or purification step is required after decontamination, and treated product can be directly returned to a patient's body or administered to a patient in need of its therapeutic effect. Therefore, pathogen reduced fluid will contain the photoproducts of the photosensitizer-like additive.

Blood or blood components to be pathogen reduced or stored include whole blood, or red blood cells, platelets and/or plasma which have been separated into components from whole blood.

The use of riboflavin and riboflavin derivatives as photosensitizers to reduce microorganisms in blood products is described in several U.S. patents, including U.S. Pat. Nos. 6,277,337, 6,258,577, 6,268120 and 6,828,323.

Pathogens which may be reduced or inactivated using the solution of this invention include any substance which is unwanted in the blood or blood components, whether originally from an external or internal source. Substances may include but not be limited to viruses (both extracellular and intracellular), bacteria, bacteriophages, fungi, blood-transmitted parasites, prions and protozoa.

Pathogens may also include white blood cells if suppression of immune or autoimmune response is desired, e.g., in processes involving transfusion of red cells, platelets or plasma when donor white blood cells may be present.

Materials which may be treated and/or stored using the methods of this invention include whole blood or separated blood components having mitochondria such as platelets.

The method of this invention for storing the whole blood or separated blood components requires mixing the riboflavin additive and the acetate with the blood component to be stored. Mixing may be done by simply adding the riboflavin and acetate in dry or aqueous form to the whole blood or blood component, or by adding a solution which contains at least the riboflavin and acetate to the whole blood or blood component to be stored. The riboflavin and acetate may be added together or each added separately.

The riboflavin additive may be used in a concentration of between about 500 μM per 35±5 mLs of solution. The concentration of acetate may be between about 140±50 mM per 35±5 mLs of solution, though wider ranges are possible. Saline containing around 0.9% sodium chloride may also be added.

If treatment to reduce or inactivate pathogens is desired, the whole blood or collected blood component containing at least the photosensitizer and perhaps acetate is exposed to photoradiation of the appropriate wavelength to activate the photosensitizer, using an amount of photoradiation sufficient to activate the photosensitizer as described above, but less than that which would cause significant non-specific damage to the blood components being illuminated or substantially interfere with biological activity of other proteins present.

If it is desired to pathogen reduce platelets, preferably the light source used to activate the photosensitizer-like additive is a broad spectrum UV light source providing light of about 320 nm.

When exposed to light, riboflavin is capable of inactivating pathogens which may be present, by interfering with the replication of the pathogens or by killing the pathogens outright. Action of the photosensitizer may be conferred by singlet oxygen formation as well as the close proximity of the photosensitizer to the nucleic acid of the pathogen and this may result from binding of the photosensitizer to the pathogens nucleic acid. “Nucleic acid” includes ribonucleic acid (RNA) and deoxyribonucleic acid (DNA). The chemistry believed to occur between 7,8-dimethyl-10-ribityl isoalloxazine and nucleic acids does not proceed solely via singlet oxygen-dependent processes (i.e. Type II mechanism), but rather by direct sensitizer-substrate interactions (Type I mechanisms). Cadet et al. [J. Chem., 23:420-429 (1983)], clearly demonstrates that the effects of 7,8-dimethyl-10-ribityl isoalloxazine are due to non-singlet oxygen oxidation of guanosine residues. In addition, adenosine bases appear to be sensitive to the effects of 7,8-dimethyl-10-ribityl isoalloxazine plus UV light. This is important since adenosine residues are relatively insensitive to singlet oxygen-dependent processes. 7,8-dimethyl-10-ribityl isoalloxazine appears not to produce large quantities of singlet oxygen upon exposure to UV light, but rather exerts its effects through direct interactions with substrate (e.g., nucleic acids) through electron transfer reactions with excited state sensitizer species. Since indiscriminate damage to cells and proteins arises primarily from singlet oxygen sources, this mechanistic pathway for the action of 7,8-dimethyl-10-ribityl isoalloxazine allows greater selectivity in its action than is the case with other photosensitizer compounds such as psoralens which possess significant Type II chemistry.

The photosensitizer-like additive and acetate may be added to or flowed into the illumination or storage container before the blood component is added to the container or may be added to the blood component which is already in the container. As noted above, the photosensitizer-like additive and acetate may also be added to the blood component as a storage solution after a pathogen reduction procedure.

For pathogen reduction procedures, the blood component to be pathogen reduced and the additive solution containing at least riboflavin are placed in bags which are photopermeable or at least photopermeable enough to allow sufficient radiation to reach their contents to activate the photosensitizer. The term “photopermeable” means the material of the container is adequately transparent to photoradiation of the proper wavelength for activating the photosensitizer-like additive. In the additive solution containing at least riboflavin, the riboflavin is added at a concentration of at least about 500 μM.

The bag containing the blood component and riboflavin is illuminated, preferably at about 1 to about 120 J/cm² for a period of between about 6 and about 10 minutes depending on the absorbtivity of the blood component being irradiated to ensure exposure of substantially all the fluid to radiation.

Acetate may be added to the blood product to be illuminated before the riboflavin is added, may be added with the riboflavin, or may be added after the illumination procedure. The acetate is added at a concentration of at least about 106 mM per 35 mL of solution. The additive solution may also contain physiological saline containing around 0.9% sodium chloride.

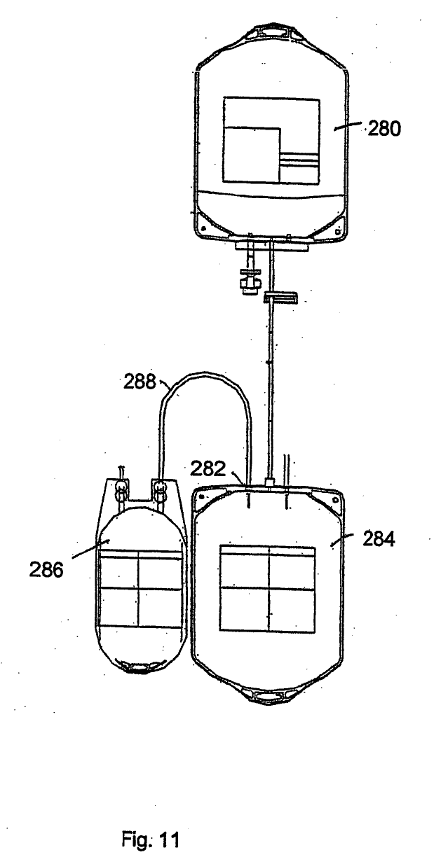

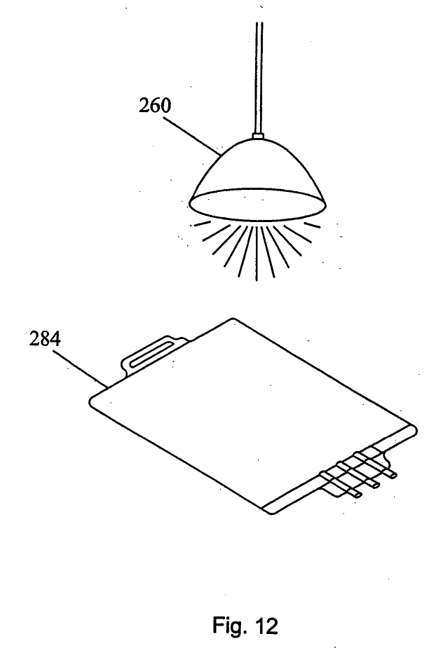

FIG. 11 depicts an embodiment of this invention in which the blood component to be pathogen reduced is initially collected in a blood bag 280. The blood component is then flowed out of collection bag 280 into a photopermeable illumination bag 284 equipped with an inlet port 282, through which riboflavin and/or acetate may be added from bag 286 via inlet line 288. Bag 284 may then be exposed to a photoradiation source 260 as shown in FIG. 12.

Alternatively, acetate may be added to the pathogen reduced blood product after the illumination procedure, and the pathogen reduced product can either be transfused immediately or stored for future use. Bag 284 could also be prepackaged to contain photosensitizer and acetate and the fluid from bag 280 may thereafter be added to the bag.

The storage solution of the instant invention also uses the additives riboflavin and acetate as described above.

### EXAMPLES

### Example 1

To measure the effect the addition of acetate has on platelets which have been subjected to a pathogen reduction procedure, platelets were suspended in solutions containing either riboflavin alone, or riboflavin and acetate and exposed to light.

These experiments include two controls, a control sample having a high concentration of platelets (150 mLs containing 3-4×10¹¹ platelets and 40 mL of plasma per 1×10¹¹ cells) (referred to as high (platelet) concentration storage in the Figures), and a standard storage control (250 mLs containing 3-4×10¹¹ platelets and 62-83 mLs of plasma/3-4×10¹¹ platelets) (referred to as standard storage control (or untreated) in the Figures).

The experiments also included two pathogen reduced platelet samples (referred to as treatments (or treated) in the Figures). One treated sample includes 3-4×10¹¹ platelets suspended in 150 mL of a pathogen reduction/storage solution containing 50 μM riboflavin and 40 mL of plasma per 1×10¹¹ cells (referred to as treatment, riboflavin in the Figures) and a sample including 3-4×10¹¹ platelets suspended in 150 mL of a pathogen reduction/storage solution containing 50 μM riboflavin and 20 mM acetate and 40 mL of plasma per 1×10¹¹ cells (referred to as treatment, riboflavin+acetate in the Figures). Both treated samples were exposed to 6.24 J/mL of light, and stored for 7 days under standard platelet storage conditions.

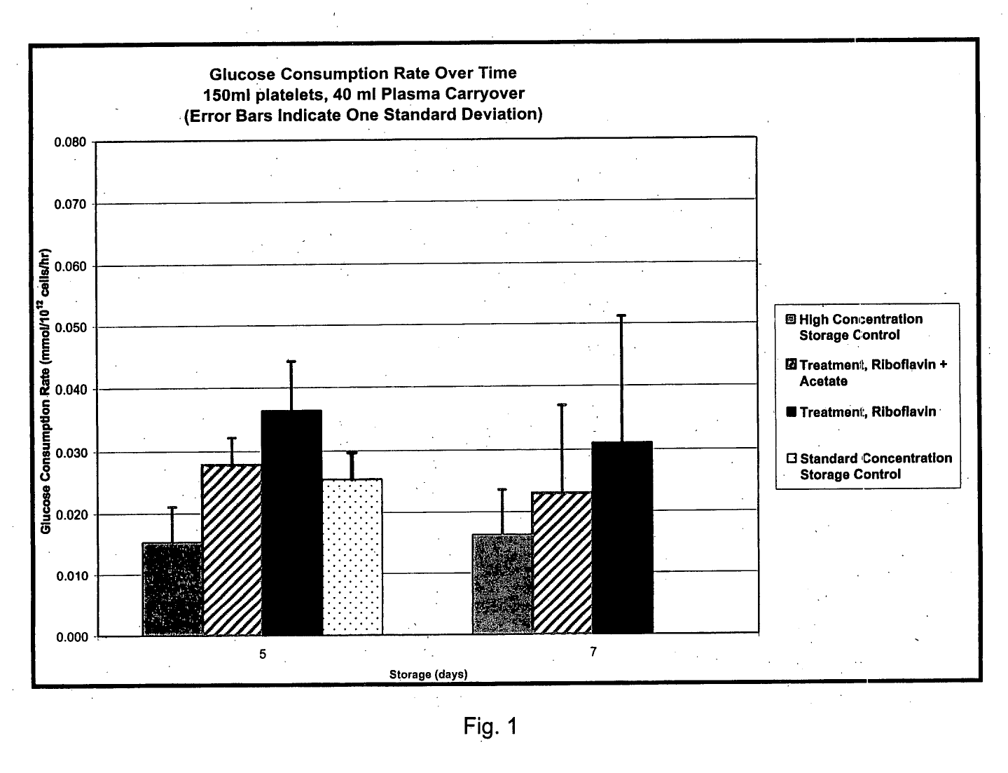

FIGS. 1-7 below show direct and indirect measurements of the metabolism of treated and untreated platelets.

FIG. 1 compares glucose consumption of treated and untreated platelets stored for 5 and 7 days. As can be seen, especially after 7 days of storage, the pathogen reduced platelets treated with riboflavin and acetate consumed less glucose than platelets treated with riboflavin alone.

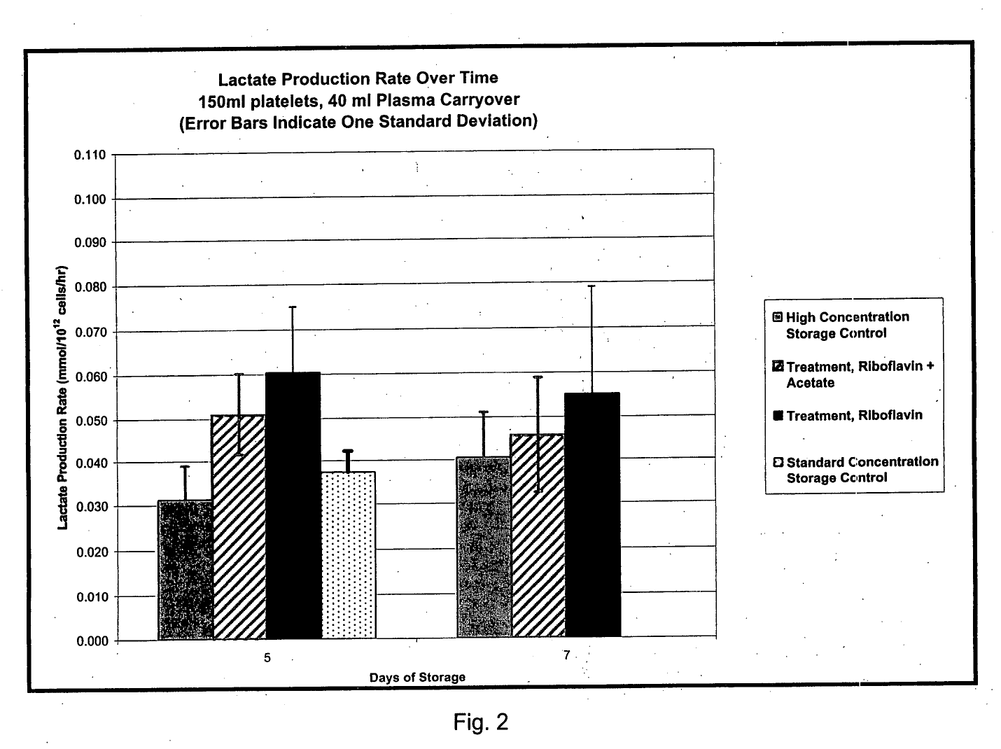

FIG. 2 compares lactate production of treated and untreated platelets stored for 5 and 7 days. Pathogen reduced platelets treated with riboflavin and acetate produced less lactic acid especially after 7 days of storage, than platelets treated with riboflavin alone.

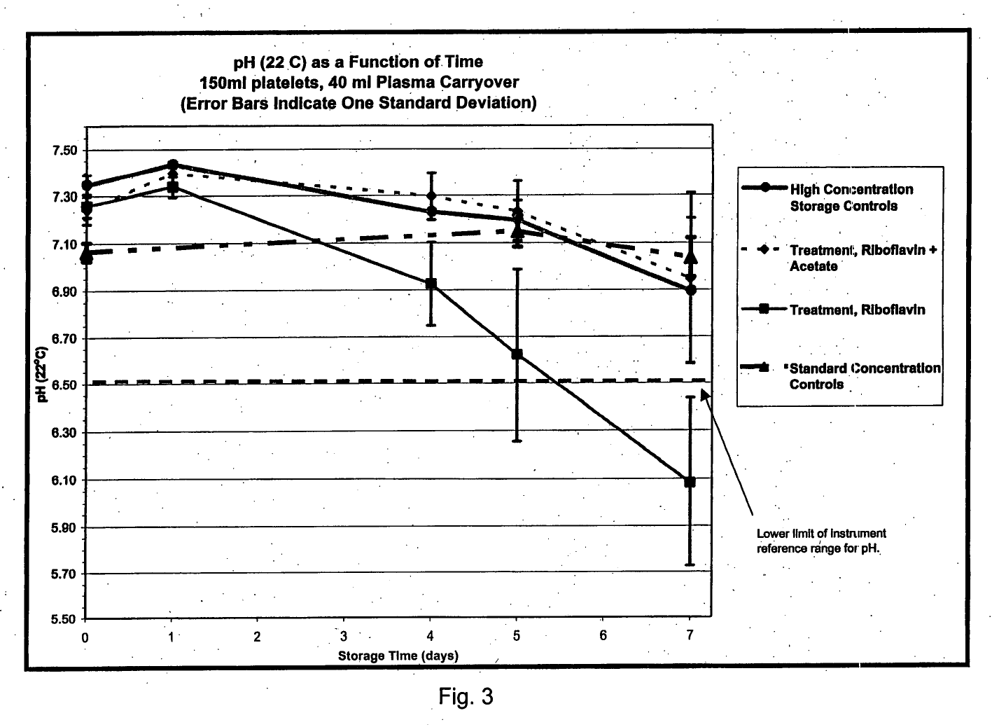

FIG. 3 compares the pH change of the pathogen reduction/storage solutions over a 7 day storage period. Pathogen reduced platelets treated with riboflavin and acetate experienced a much slower change (or drop) in pH of the pathogen reduction/storage solution over the 7 day storage period. At day 7, the average pH is above 7.0. For platelets in pathogen reduction/storage solution without acetate, the pH is below 6.8.

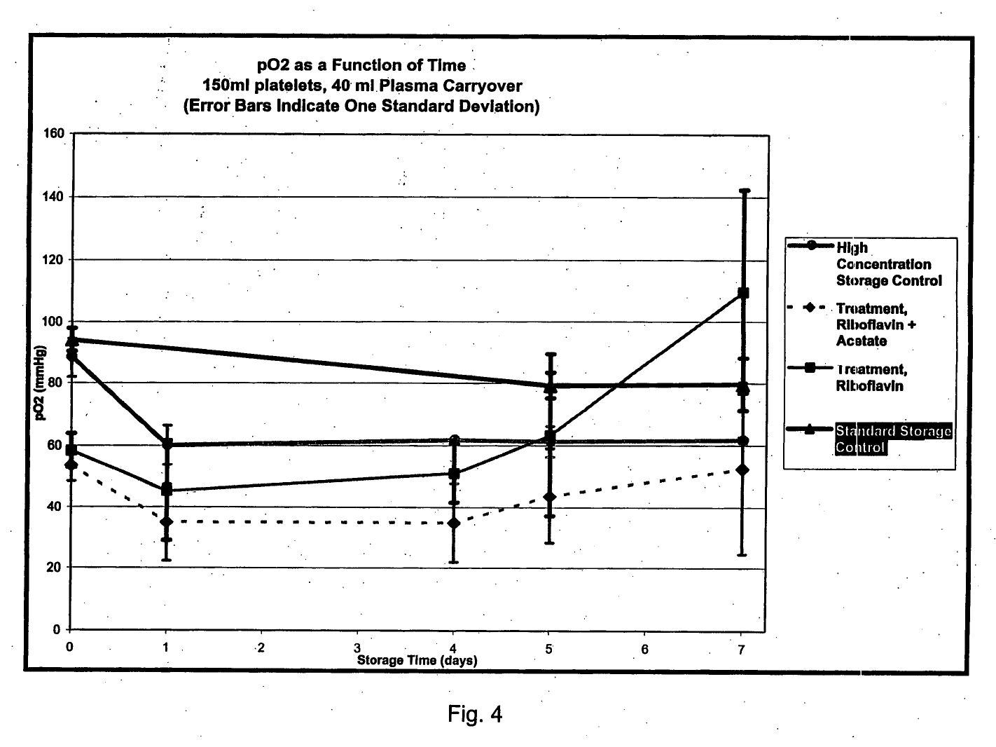

FIG. 4 compares the consumption of oxygen of the pathogen reduced platelets over a 7 day storage period. Oxygen consumption continually increased during the 7 day storage period by pathogen reduced platelets treated with riboflavin and acetate as well as riboflavin alone, as compared to both sets of control platelets. Oxygen consumption is indicative of mitochondrial respiration. Lower values of pO₂ reflect higher oxygen consumption and better mitochondrial activity.

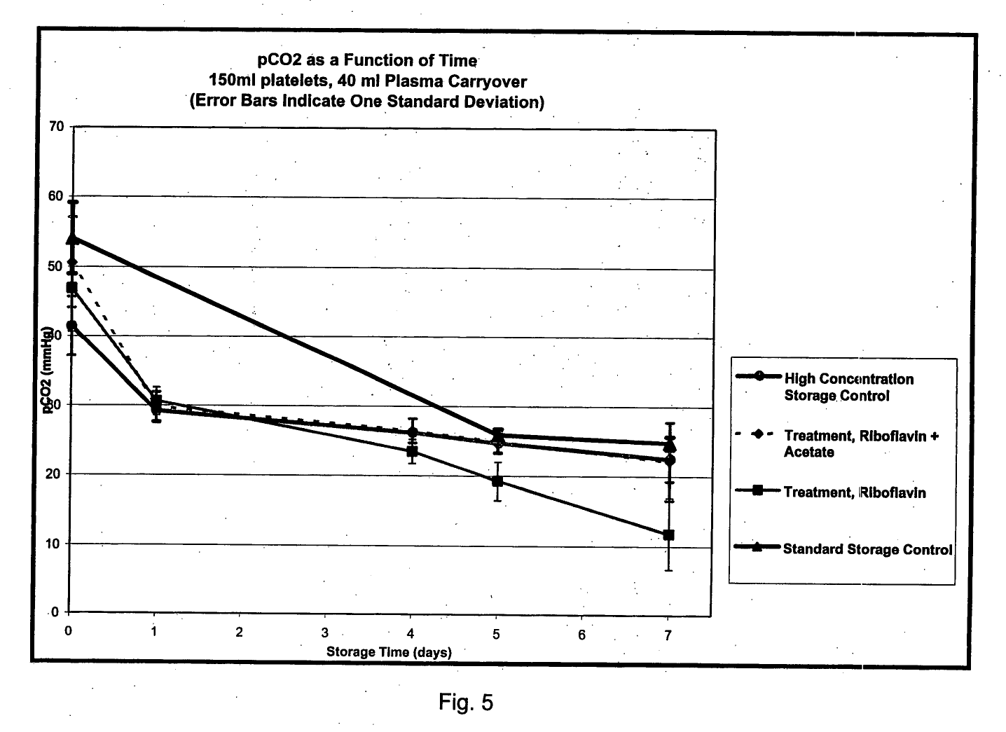

FIG. 5 compares carbon dioxide production by platelets over 7 days of storage. Carbon dioxide production is a measure of mitochondrial respiration; respiring platelets consume oxygen and produce carbon dioxide. More carbon dioxide is produced by pathogen reduced platelets treated with riboflavin and acetate, than by control untreated platelets.

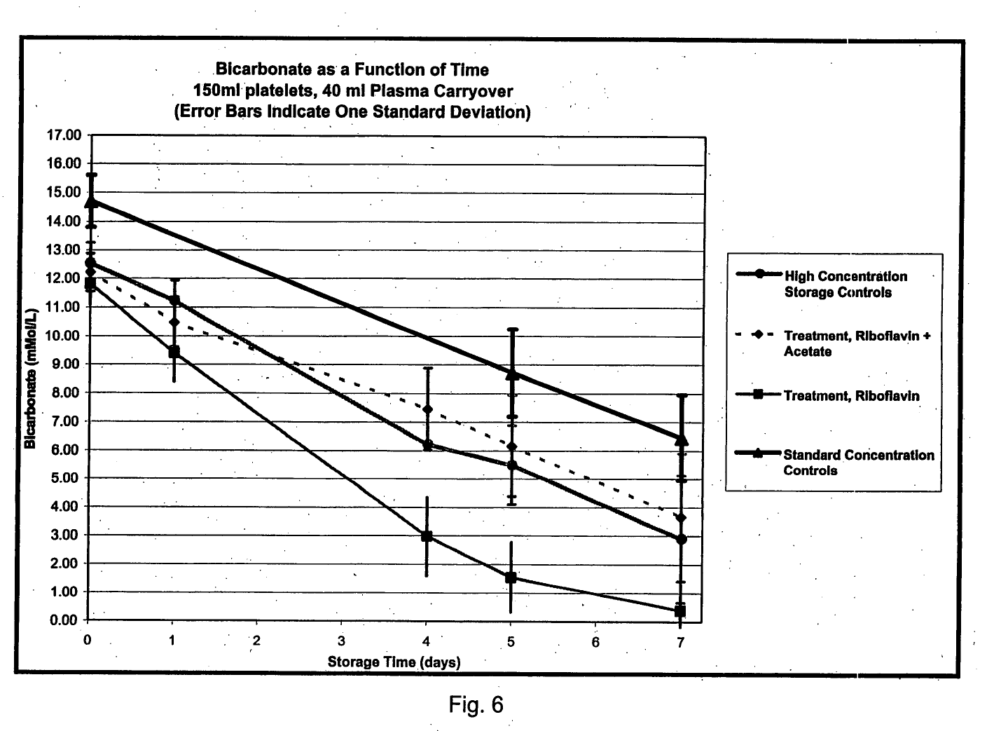

FIG. 6 compares the neutralization of bicarbonate by platelets in 40 mL plasma carryover in the pathogen reduction/storage solutions over 7 days of storage. Platelets metabolize bicarbonate to maintain a constant pH. If the pH drops due to production of lactic acid, more bicarbonate will be neutralized. Pathogen reduced platelets treated with riboflavin and acetate neutralized less bicarbonate than control untreated platelets.

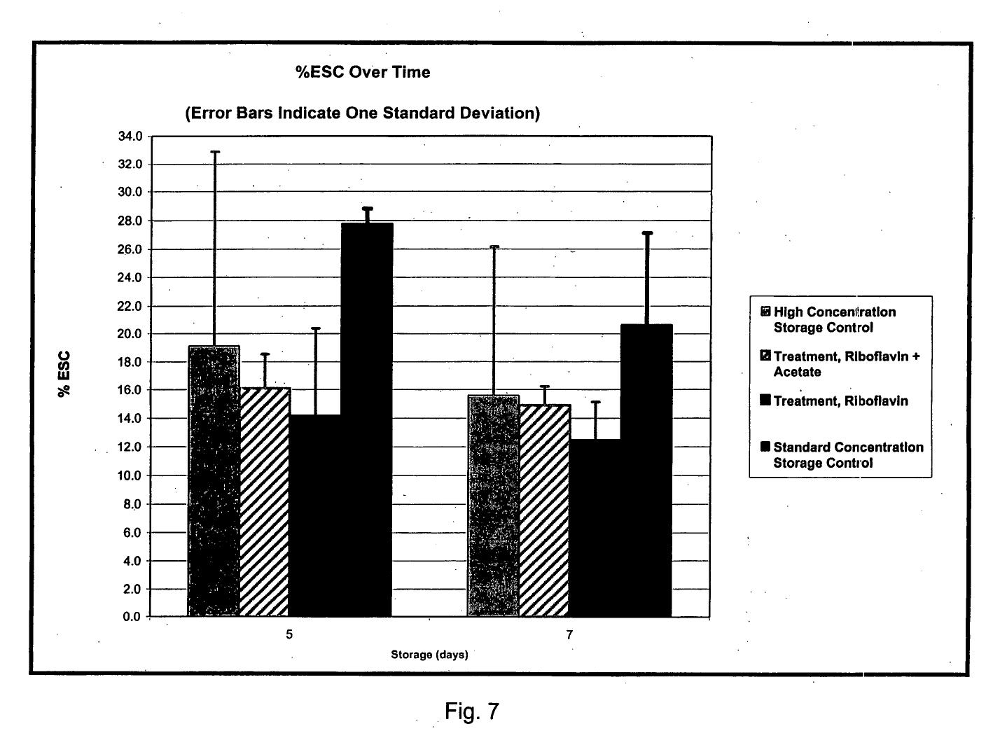

FIG. 7 compares the percentage of extended shape change of platelets between 5 and 7 days of storage. Again, platelets treated with riboflavin and acetate showed less shape change after 7 days in storage, than platelets treated without acetate.

As can be seen in FIGS. 1-3, the addition of acetate produces significant improvements in glucose consumption, lactic acid production and pH, which are the most predictive indicators of platelet recovery and survival in vitro. This effect is consistent with acetate in combination with riboflavin promoting mitochondrial respiration.

This data also shows that an additive solution containing riboflavin and acetate allows for storage and/or pathogen reduction of high concentrations of platelets while decreasing plasma concentration. This allows more plasma to be collected in a blood separation procedure and decreases plasma exposure levels in a transfusion recipient.

### Example 2

A comparison study was done to look at the effect of acetate on platelets stored for 12 days. The platelets were not exposed to light.

One set of samples containing 250 mL platelets at a concentration of 900-2100×10³/μL was suspended in 35 mL of a storage solution containing saline with 1.85 M sodium acetate and 500 μM riboflavin.

The other sample containing 250 mL platelets at a concentration of 900-2100×10³/μL was suspended in 37 mL of a storage solution containing saline only.

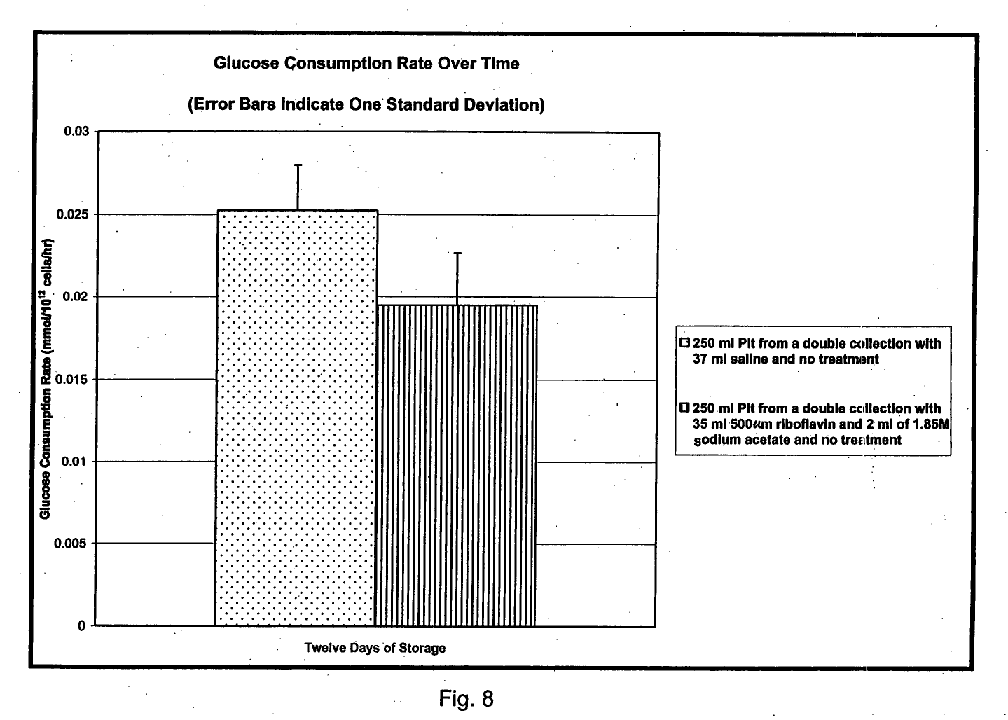

FIG. 8 compares the rate of glucose consumption by platelets stored in a solution containing riboflavin and acetate with platelets stored in a solution without riboflavin and acetate. After 12 days of storage, platelets in a solution containing riboflavin and acetate consumed less glucose than platelets stored in a solution without riboflavin and acetate.

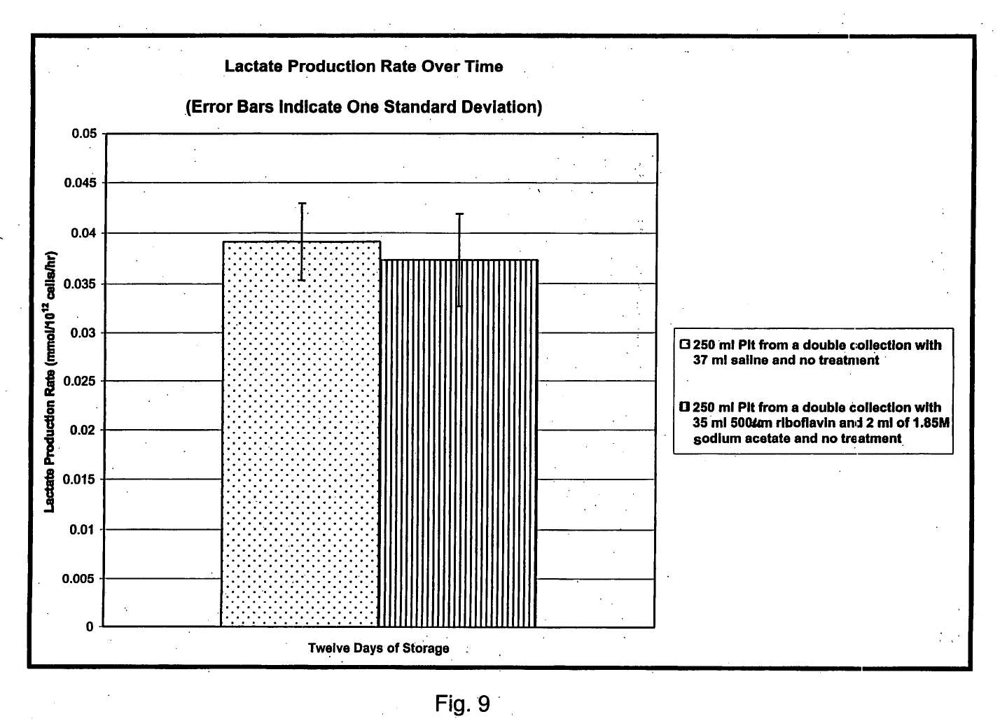

FIG. 9 compares the rate of lactate production by platelets after 12 days of storage. After 12 days of storage, platelets in a solution containing riboflavin and acetate produced less lactic acid than platelets stored in a solution without riboflavin and acetate.

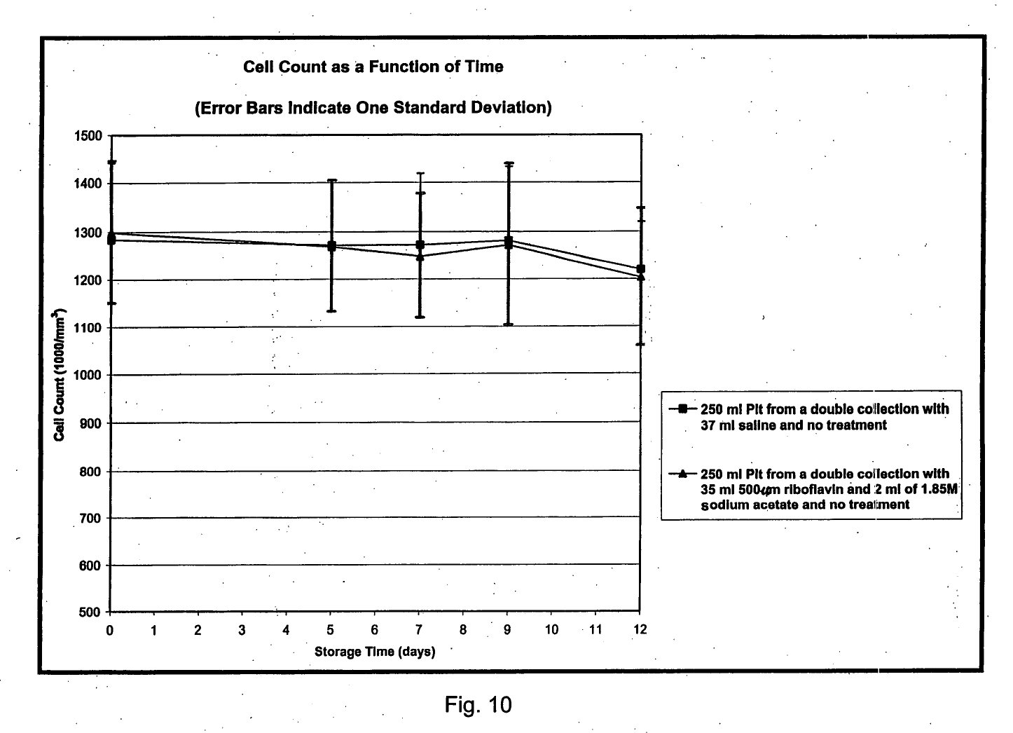

FIG. 10 compares the cell count of platelets stored in a storage solution containing riboflavin and acetate with the cell count of platelets stored in a solution without riboflavin and acetate. Over 12 days of storage, there appears to be no measurable effect on the cell count for platelets stored in a solution containing riboflavin and acetate vs. platelets stored in a solution without riboflavin and acetate.

The results indicate the benefit of using a storage solution containing riboflavin and acetate. As can be seen in FIGS. 8-10, storage of platelets in a solution containing both acetate and riboflavin enables storage of platelets for at least 12 days, as compared to platelets stored in solutions without riboflavin and acetate.

1. A fluid comprising:
at least one collected blood component; and
a blood component additive solution comprising
an endogenous alloxazine, and
acetate.

2. The fluid of claim 1 wherein the endogenous alloxazine is riboflavin.

3. The fluid of claim 1 wherein the at least one collected blood component comprises platelets.

4. The fluid of claim 1 wherein the blood component additive solution further comprises physiological saline.

5. The fluid of claim 4 wherein the physiological saline is 0.9% sodium chloride.

6. The fluid of claim 3 further comprising plasma.

7. The fluid of claim 6 wherein the volume of plasma is between 20-80 mL per 10¹¹ collected platelets.

8. The fluid of claim 6 wherein the volume of plasma is between 30-60 mL per 10¹¹ collected platelets.

9. The fluid of claim 1 wherein the at least one collected blood component has been pathogen reduced.

10. A storage or additive solution comprising:
an endogenous alloxazine; and
acetate.

11. The storage or additive solution of claim 10 wherein the endogenous alloxazine is riboflavin.

12. The storage or additive solution of claim 10 further comprising physiological saline.

13. The storage or additive solution of claim 12 wherein the physiological saline is 0.9% sodium chloride.

14. A storage or additive solution consisting essentially of:
an endogenous alloxazine; and
acetate.

15. The storage or additive solution of claim 14 wherein the endogenous alloxazine is riboflavin.

16. The storage or additive solution of claim 15 wherein the riboflavin is in a concentration of about 500 μM per 35±5 mLs of solution.

17. The storage or additive solution of claim 14 wherein the acetate is in a concentration of around 140±50 mM per 35±5 mLs of solution.

18. A storage or additive solution consisting of:
riboflavin;
acetate; and
saline.

19. A fluid which has been pathogen reduced consisting essentially of:
collected blood or blood components; and
a pathogen reduction solution consisting essentially of
photoproducts of a photosensitizer-like additive;
acetate; and
saline.

20. The fluid of claim 19 wherein the collected blood or blood components further consists essentially of platelets and plasma.

21. The fluid of claim 20 wherein the plasma is between 20-80 mL per 10¹¹ collected platelets.

22. The fluid of claim 20 wherein the plasma is between 30-60 mL per 10¹¹ collected platelets.

23. The fluid of claim 19 wherein the photoproducts of a photosensitizer-like additive are photoproducts of an endogenous photo sensitizer.

24. A pathogen reduction solution comprising:
an endogenous alloxazine; and
acetate.

25. The pathogen reduction solution of claim 24 further comprising saline.

26. The pathogen reduction solution of claim 24 wherein the endogenous alloxazine further comprises riboflavin

27. A pathogen reduction solution consisting of:
riboflavin;
acetate; and
saline.

28. A method of pathogen reducing blood or collected blood components which may contain pathogens comprising:
(a) mixing an effective non-toxic amount of a mixture consisting essentially of an endogenous photosensitizer and acetate with the blood or collected blood component to make a mixed fluid; and
(b) exposing the mixed fluid to photoradiation sufficient to activate the photosensitizer whereby at least some of the pathogens are reduced.

29. The method of claim 28 wherein the collected blood component comprises platelets.

30. The method of claim 28 further comprising adding physiological saline to the mixed fluid.

31. The method of claim 29 wherein the mixed fluid further comprises plasma in an amount between 20-80 mL per 10¹¹ collected platelets.

32. The method of claim 29 wherein the mixed fluid further comprises plasma in an amount between 30-60 mL per 10¹¹ collected platelets.
